(19) United States
(12) Patent Application Publication
(10) Pub. No.: US 2013/0226038 A1
(43) Pub. Date: Aug. 29, 2013

(54) DEVICE AND METHOD FOR MEASURING AND ASSESSING MOBILITIES OF EXTREMITIES AND OF BODY PARTS

(71) Applicant: SportMed AG, (US)

(72) Inventor: Christian Egen, Bitburg (DE)

(73) Assignee: SportMed AG, Bitburg (DE)

(21) Appl. No.: 13/780,537

(22) Filed: Feb. 28, 2013

(30) Foreign Application Priority Data
Feb. 28, 2012 (DE) 102012202990.7

(51) Int. Cl. A61B 5/11; A61B 5/00

(52) U.S. Cl. CPC A61B 5/11; A61B 5/0004; A61B 5/72; A61B 5/6813
USPC 600/595

(57) ABSTRACT

A device and method for measuring and assessing mobilities of extremities and body parts of a proband, characterized by a wireless measuring device attachable at an extremity to be measured or at a body part to be measured of the proband, the measuring device including a sensor for three-dimensional continuous detection of position changes of the measuring device, a data processing device that wirelessly receives the measured values, an input device for manual inputs, wherein the input device is part of the wireless measuring device, an analysis program for a planning of a measurement and/or for a visualization of a measurement and/or for an analysis of a measurement, wherein the analysis program runs on the data processing device and the analysis program computes from the measured values position changes of the measuring device, computes from the position changes a mobility of an extremity or of a body part; and processes manually inputted inputs.

This application claims priority to German Patent Application No. 10 2012 202990.7 filed Feb. 28, 2012, this application being fully incorporated herewith by reference.

### 1. TECHNICAL FIELD

The present invention relates to a device and a method for measuring and assessing mobilities of extremities and of body parts of a proband, typically of a human. Mobilities of extremities of a proband are measured in the diagnostics of a medic or of a physiotherapist, in order to provide information about the mobility of specific joints of a patient. Then, this information can be used as starting point or for supervising of target-oriented therapy measures.

### 2. PRIOR ART

The physical examination of joints and body parts concerning mobility and concerning the quality of the mobility is part of the thorough examination of the muscular skeletal system and is often named as “manual inspection”. This inspection is divided into quantitative measuring of the range of movement of a joint or of a body region as well as into the qualitative examination of the motivity as well as its documentation. Both aspects of the physical examination of a body region require an appropriate positioning of the patient and an isolated movement of the examined region.

During the physical examination of the movement quality and motility, the corresponding body region is examined in consideration of status, pain and muscular motility, qualitatively described and documented. The qualitative detection of the muscle function is described in general by attributes of in-mobility via degrease up to above-average motility (force). The examination is based herein on the personal evaluation of the examiner.

Examples for such ways of description can be found amongst others in
JANDA, V.: Manuelle Muskelfunktionsdiagnostik. Munich: Urban & Fischer publishing GmbH/Elsevier GmbH, 4^{th} Ed., 2009.

and
KENDALL, F. P.: Muskeln—Funktionen und Tests. Munich: Urban & Fischer publishing GmbH/Elsevier GmbH, 4^{th} Ed., 2001.

The results of an examination are documented by the examiner, what leads to a permanent change between documentation and examination at the patient. It is also common practice, to dictate the results to an assistant. Indeed, this possibility is fast in the execution, but is combined with economic disadvantages and the risk of wrong understanding during the dictate.

The extremities are in particular the arms, the legs and the head of a proband and patient, respectively. With devices for measuring mobilities of extremities, not only the mobilities of the joints between the torso and the extremity can be measured, but also of joints between two parts of an extremity, for instance the mobility of an elbow- or knee-joint.

Mobilities of extremities of patients or probands can be for instance determined and analyzed according to the neutral-zero-method. Starting point of a measuring according to the neutral-zero-method is the so called neutral-zero-position, wherein the body of the proband is in the following defined starting position:
upright standing with forward-facing direction of view;
the arms hang relaxed downwards at the side of the body, wherein the palms of the hands are directed towards the thighs;
the feet stand in a hip-width distance and parallel.

The angles of the extremities to the torso and to the other parts of the extremities that result in this position are defined as zero-position.

During a measuring, the examiner moves an extremity of the proband from the zero-position to an end-position, which describes the specific position, from which the extremity cannot be moved farther anymore in the direction of movement. Then, the angle between zero-position and end-position is measured.

The measuring is carried out starting from the zero-position in both directions of movement, which shall be in one plane. At the example of the knee, also the maximum stretching and the maximum flex of the joint of the knee is measured.

The measuring results according to the neutral-zero-method are indicated in form of a number-triple. In the example of measuring a joint of a knee for instance results the following: “5-0-110”. This result says that a proband is able to extend his/her knee out of the zero-position up to 5°, i.e. to stretch it, and to flect it up to 110°, i.e. to bow it.

A continuative description of the neutral-zero-method is given in the book:
MEINECKE, R.; GRÄFE, K.: Bewegungs-, Längen und Umfangsmessungen: Neutral-Null-Durchgangsmethode. Haan: Verlag Europa-Lehrmittel Nourney, Vollmer GmbH & Co. KG, 2007.

A common device for measuring mobilities of extremities according to the neutral-zero-method is a goniometer. A goniometer is a mechanical angle-measuring instrument with two moveable arms between which a scale is arranged for measuring an angle. This device comprises the disadvantage that it comprises a high measurement uncertainty. This is caused in particular in the fact that common goniometers comprise comparatively short arms in comparison to the length of for instance an arm or a leg of the proband. Goniometers with long arms are indeed more precise, but substantially more difficult to handle. A further problem is the missing supervision of the correct execution of the movement. In addition, common goniometers do not comprise a possibility to transfer data for analysis matters directly to a data processing device.

This problem is solved since some years by in parts complex electro-mechanic frames for the fixation and guidance of the extremity of a proband in combination with a data processing device.

So, for instance patent application publication DE 39 07 140 A1 comprises a measurement device for the determination of the active and passive mobility of a shoulder joint. The measurement device consists of an electro-mechanical unit and a computer-controlled unit. The electro-mechanical unit is rigidly connected to the arm of the patient in that the arm might direct in every possible three-dimensional angle, which is possible for the shoulder joint. The electro-mechanical unit comprises two rotation axes that are coupled together via a bracket, wherein the extensions of the axes meet in the center of the upper arm head, which is considered to be a ball.

From patent document AT 384 544 B a method is known for the determination of the mobility of body parts by subsequent position measuring by means of electronic measurements. Therefore, at one or more body parts ultrasonic receivers or ultrasonic senders are applied, which work together with assigned ultrasonic receivers or ultrasonic senders, which are located in fixed positions in the three-dimensional space.

It is disadvantageous for such arrangements, that they are in their entirety or at least in view of the components which are fixed in the three-dimensional space, in their dimensions very large and can only be difficultly transported. Thus, they can only hardly be used during physiotherapeutic home visits. Furthermore, such arrangements are very limited in their usability, since they are in particular designed for the measurement of one very specific motion sequence, but are not suited for the use as general measuring device for the execution of a plurality of mobility measurements.

From the patent application publication DE 102 14 318 A1 an angle measuring device and an angle measuring method for mobility control of muscles and/or joints is known, that are responsive for the extremities of a human. The angle measuring device comprises herein on the one hand an electronic angle measuring sensor for the detection of an angular position of vectors that lie in an in general vertical plane. The angle sensor is herein attached to an extremity or at the torso of a proband.

It is also disadvantageous in the system, that inputs before and after the measurement have to be carried out preferentially via input means of the data processing devices. In order to carry out inputs, the examiner has to leave the proband between the measurements. This is considered to be very uncomfortable and time intensive. Furthermore, input means like keyboards etc. of data processing devices are not usable in the clinic use, since they cannot sufficiently kept sterile.

In addition, such measuring systems, which base on one single sensor, are very prone to measuring deviations, since there is no possibility to check the measurements. In addition, during the execution of repeated measurements deviations come up due to the versatile variation options that these deviations are in most cases higher than the deviations that are induced during a single measurement by the sensor. Consequently, the examining person has to rely on the result of a single measurement, which comprises significant uncertainties.

Thus, it is the problem of the present invention to provide a device for measuring and assessing mobilties of extremities and of body parts and a corresponding method for measuring and assessing mobilities of extremities and of body parts, which comprise the following advantages:
Generation of measured values of the mobility examination with a significantly reduced measurement uncertainty;
Improvement of handling comfort and usability for the examiner and the proband;
Abetting the clinical use by improved hygiene; and
Support of the examiner during the measurement and documentation both for single measurements and for entire complex measurement problems.

### 3. SUMMARY OF THE INVENTION

The above-mentioned problems are solved by a device for measuring and assessing mobilities of extremities and of body parts according to embodiments of the present invention as well as by a method for measuring and assessing mobilities of extremities and of body parts according to embodiments of present invention.

In particular, the above-mentioned problems are solved by a device for measuring and assessing mobilities of extremities and of body parts of a proband comprising a wireless measuring means that is attachable or fixable at an extremity to be measured or at a body part to be measured of the proband, wherein the measuring means comprises at least one sensor for a three-dimensional continuous detection of position changes of the measuring means, a data processing device that wirelessly receives the measured values of the measuring means, an input means for manual inputs by a user, wherein the input means are part of the wireless measuring means, an analysis program for a planning of a measurement and/or for a visualization of a measurement and/or for an analysis of a measurement, wherein the analysis program runs on the data processing device and the analysis program computes from the measured values of the measuring means position changes of the measuring means, computes from the position changes a mobility of an extremity or of a body part and processes inputs of the user manually inputted via the input means.

By means of the measuring device according to the invention mobilities of extremities or of body parts of a proband or patient can be measured, when in a normal case, a person, which examines the proband, attaches the wireless measuring means at the extremity to be measured and then moves this extremity together with the measuring means in the plane to be measured. Of course, the measuring device according to the invention is also appropriate for the measuring of other movements of the body and of body parts. Also a measurement is possible, wherein the proband moves the extremity or the body part himself/herself. Herein, the wireless measuring means can be fixed at the extremity or at the body part.

The at least one sensor detects continuously a three-dimensional relative movement of the wireless measuring means. Besides three-dimensional sensors, which are able to detect a relative movement in all three space dimensions themselves, also a plurality of one-dimensionally or two-dimensionally measuring sensors can be used, which detect together continuously a three-dimensional relative movement of the wireless measuring means. By the measurement of a relative movement by corresponding sensors, fixed points or fixed sensor components etc. can be omitted. Therefore, it is sufficient to use one single wireless measuring means according to the invention together with a corresponding data processing device in order to execute a precise measurement. The measuring device is thus all in all small and handy and is also appropriate for the mobile use.

As soon as at least one measurement (for example a change in height) is determined by at least two sensors at the same time and respectively in parallel, it is possible to generate for this measurement a mean value. This mean value can either be computed by the measuring means itself or after transmission of the raw data in the data processing device by the analysis program. This determination of a mean value allows compensating random deviations and measurement uncertainties of the sensors.

Furthermore, before the mean value generation is executed, a plausibility check is done, which avoids that falsified measured values, like for instance caused by irritations of the magnetic field sensor due to magnetic metal masses, influence the measurement.

If moreover sensors are used for the parallel measurement, which base on different effect principles (acceleration, magnetic field, gravity field, position change), it is possible, to compensate besides the random deviations of the sensors also deviations that are caused by the measuring principles.

As data processing devices, for instance, commercial stationary computers or portable systems can be used. Portable systems, like for instance notebooks, subnotebooks or tablet-PCs are in particular then advantageous, when the measuring device shall be used for home visits to patients and respectively to probands.

A further advantage of the present invention lies in the significantly increased handling comfort for the person who examines the proband, i.e. for the examiner. The measuring means is wirelessly connected with the data processing device so that the free-moving space of the examiner and the proband is not limited by cables etc. Preferably, the measuring means is supplied by an internal current source, like for instance a battery or an accumulator with the necessary electrical energy, and then does not need to be connected to an external current source.

The measuring means comprises input means for manual inputs by a user, normally the examiner or rarely the proband itself. Preferably, all functions that are necessary for a measurement of an analysis program, which is executed on the data processing device, can be controlled by the input means. Herewith, the input means allow the examiner to stay also during an entire measurement sequence of a measurement profile with the proband. The examiner does not have to leave the proband during the execution of a measurement of a measurement sequence, in order to do possible inputs at the data processing device.

It is in particular preferred, to be able to carry out all inputs, which are necessary for planning, execution and analysis of the measurement, and the navigation in the analysis program via the input means of the wireless measuring means. In particular also subjective or qualitative assessments can be inputted. These qualitative assessments can be additional assessments like for instance the pain feeling or the end feeling of the proband or further examination assessments of the manual joint inspections, like for instance the force of the examined musculature.

Such qualitative assessments can be inputted by the user directly during or after the quantitative measurement via the input means of the wireless measuring means. Thus, the objective mobility measurement and the subjective assessments are carried out by the same measuring means and in one step, what significantly simplifies the examination of the proband and accelerates it.

An additional advantage lies in the increased hygiene during the measurement of mobilities of extremities. The measuring means is preferably attached during the measuring by the examiner directly to the extremity to be measured of the proband. By the wireless form, the measuring means can be designed in that it can be simply disinfected and respectively sterilized. This can be ensured by the constructive design of the measuring means, by the selection of the used materials and by the provision of specific sealing members.

A further hygienic advantage results by the fact that the examiner can carry out all necessary inputs via the sterile measuring means itself during the measurements. Thus, the examiner does not have to carry out inputs during the measurements via non-sterile input means, like keyboards and the like of the data processing device. Herewith, a transfer of viruses and bacteria to the proband and from the proband is avoided. Thus, the measuring device according to the invention allows measurements with significantly reduced measurement uncertainty, an optimum mobility of the measurement device in its entirety, a significantly increased handling comfort during the care of the patient and measurements with increased hygiene. By the chosen sensors, which completely waives external reference points, which have to be set up, or markers, advantages in view of mobility, manufacturing costs and initial efforts before the measurement are achieved.

Preferably, the sensor comprises a three-dimensional magnetic field sensor and/or a three-dimensional gyroscope and/or a three-dimensional acceleration sensor in order to detect position- and orientation-changes of the wireless measuring means. By means of a three-dimensional magnetic field sensor, the orientation of the measuring means corresponding to the magnetic field of the earth or to other non-natural magnetic fields can be measured in all three space dimensions. A three-dimensional gyroscope measures angular accelerations of the measuring means around all three main axes, which are caused by rotations of the measuring means. By means of the three-dimensional acceleration sensor, linear accelerations of the measuring means in all three main axes can be measured. Correspondingly, by the acceleration sensors, inclines of the measuring means to the gravity field of the earth are measured.

Preferably, a measuring means according to the invention comprises both a three-dimensional magnetic field sensor and a three-dimensional gyroscope and a three-dimensional acceleration sensor. Since all three kinds of sensors detect three-dimensional relative movements, some of the measured values or all of the measured values are overdetermined. From these measured values of the different sensor types an orientation vector is computed, by means of a so called “strapdown-calculation” and by means of a so called “kalman-filter” and then thereof the position and orientation change of the measuring means is computed. This calculation can already take place in the measuring means itself or in the analysis program or in parts in both components.

Since the measured values of the position changes are overdetermined, random deviations of the sensors and other measurement failures like completely non-plausible measured values, can be compensated, what leads all in all to a very precise position- and orientation-determination of the measuring means relative to the start position.

Preferably, a plurality of measuring means is wirelessly connected to the data processing device. The data processing device is able to receive and to process the data of a plurality of measuring means, like for instance of four measuring means. Herewith, it is possible to examine twin movements of the proband. Herewith, every measuring means can identify itself definitely in due to the data processing device and the analysis program so that the received measured values can be assigned definitely to the corresponding measuring means. Such a measuring means can for instance be used in order to develop and to use new methods for mobility- and posture-measurements—which exceed the neutral-zero-method.

Preferably, the input means are designed for multi-dimensional inputs, in particular for the navigation in the analysis program or for the input of assessments. Such input means for multi-dimensional inputs maybe designed for instance as a control-cross, an arrangement of four direction pointers and a confirm button or as a touch sensitive surface for the interaction of the examiner with the measuring means.

Preferably, the sensor detects the incline of the measuring means and the analysis program computes from the incline manual input values of the user. Herewith, the user can input for instance values for the pain feeling during a movement on a scale of for instance 1 to 10 by simple and intuitive incline of the measuring means.

Preferably, the sensors do not need any reference points in the measuring field. By reference points, fixed points in space are meant, which are necessary in the prior art for the determination of an absolute position of the measuring means. The present measuring means does in a preferred embodiment not need corresponding fixed points, since the determination of an absolute position of the measuring means is not necessary. Contrary to that the relative position and facultatively the relative orientation of the measuring means in view of a start point is used. For the analysis of mobility of its extremities—like for instance according to a neutral-zero-method—the zero position of the extremity is the relative start point. This relative measurement has the advantage that no additional components, like for instance signal sources, which are fixed in space, or sensors, which are fixed in space, have to be set up and calibrated for the execution of a measurement. This is in particular advantageous during mobile use.

Preferably, the manual inputs, which are possible at the wireless measuring means, enclose an identification of a zero-position of the extremity and/or of an end-position of the extremity and/or noting a subjective assessment concerning the proband during the measurement. By means of the above mentioned inputs, it is possible to execute fundamental inputs, which have to be carried out during a measurement, directly via the input means of the measuring means. Thus, the examiner can stay during the execution of the measurement at the proband and does not have to see-saw between the proband and data processing device. Furthermore, it is ensured that the subjective assessments are assigned to the correct measurement, i.e. to the correct joint.

Preferably, the analysis program comprises a teach-in-module for the calibration of the measurement plane before executing the real measurement. For the supervision of the correct movement sequence of the extremity of the proband it can be necessary to teach-in the measuring device the correct movement sequence. The teach-in is in particular not for the sensoric calibration, but the determination of the plane, which is relevant for the measurement, i.e. the plane, in which the movement shall be measured. During teach-in, the measurement device determines from the defined executed movement sequence the plane of the subsequent measurement. The teach-in-module instructs the examiner to comply with the correct movement sequence.

Preferably, the analysis program comprises a pool of pre-defined single measurements, which can be combined by the analysis program to a measurement profile. Herein, the analysis program provides during the measurement preparation a plurality of pre-defined single measurements, which are combined in a pool. Out of this pool, which contains in particular all standard-single measurements of the neutral-zero-method, the examiner chooses the single measurements, which have to be executed, and arranges them in the analysis program to an individual measurement profile for this specific proband. Herewith, the real measurements at the proband can be executed uninterruptedly one after the other. In addition, recurring measurement profiles can be saved.

In the context of the measurements, information about the joint to be examined, the movement direction to be examined, information about the specific neutral-zero-position of the joint to be examined, information about a teach-in-measure, which has to be executed, requirements of the analysis of the measurement, graphical illustrations for the visualization of the movement sequence and pre-defined input options for additional subjective assessments can be displayed by the analysis program to the examiner. During the real measurement, a graphical illustration of the extremity can be displayed by the analysis program, which moves simultaneously to the movement of the real extremity, in order to signalize the examiner the measurement progress and a successful measurement.

Furthermore, the problem according to the invention is solved by a method for measuring and assessing mobilities of extremities and of body parts of a proband, comprising the following steps:
a. providing a wireless measuring means that is attachable or fixable at an extremity to be measured or at a body part to be measured of the proband, wherein the measuring means comprises sensors for the three dimensional continuous detection of position changes of the measuring means;
b. providing a data processing device, which receives the measured values of the measuring means wirelessly;
c. providing an input means for manual input by a user, wherein the input means is part of the wireless measuring means;
d. attaching or fixing the measuring means at the extremity to be measured or at the body part to be measured of the proband;
e. moving the extremity of the proband;
f. manually inputting a subjective assessment of the proband via the input means; and
g. analyzing the measurement by the analysis program.

Thus, the method according to the invention allows for the above already explained reasons in the context of the measuring device also measurements with significantly reduced measurement uncertainty, an optimum mobility of the entire measuring device, a significantly increased handling comfort and an improved care of the patient and measurements with improved hygiene. Moreover, by the measurement of position changes of the measuring means complex fixed points or stationary reference points of the sensors are omitted, what makes the measuring device all in all significantly cheaper. Preferably, for the execution of the method, a measuring device according to patent claim 1 is used.

In particular, now, subjective assessments can be inputted directly via the wireless measuring means and can be thus documented. The objective mobility measurement and the subjective assessment can be executed by the same measuring means and in one step, what significantly simplifies the examination of the proband and accelerates it.

Preferably, the method moreover comprises a step of choosing the measurement to be executed in an analysis program out of a pool of pre-defined single measurements. The examiner can in advance select single measurements to be executed from the pool and combine these to an individual measurement profile for the proband. Herewith, the efficiency during the real measurement of the proband can be increased, by offering the single measurements one after the other by the analysis program and can be executed.

Preferably, the user can navigate in the analysis program by means of the input means of the wireless measuring means or can choose the measurements to be executed. The examiner is able to do inputs for the analysis program directly at the wireless measuring means, without having to leave the proband during the execution of the measurement.

Preferably, the method comprises furthermore a teach-in-step, by which the movement plane to be measured is recognized and the analysis program is calibrated. With the teach-in-step, the plane, which is foreseen for the measurement is taught to the analysis program by defined movement in the plane.

Preferably, the analysis step comprises a partial step of a continuous computing of the relative position and relative orientation in space of the measuring means out of a plurality of overdetermined measured values of three-dimensional sensors. Herewith, in particular the end-position and the end-orientation of the extremity are computed. By the computing of the end-position and the end-orientation on the basis of overdetermined measured values, the measurement precision is very high despite of measuring without locationally fixed reference points. The start- and the end-position of the extremity or of the body part can be on the one hand determined by a manual input at the measuring means or they can be derived after the complete movement directly from the extremes of the continuous measurement.

Preferably, the method comprises furthermore a step of numeric and/or graphic real time-output of the measuring results during the measurement on a screen of the data processing device. Herewith, the examiner sees on a screen of the data processing device in real time, how the extremity of the proband moves during the measurement and whether the measurement proceeds prosperously. If the extremity leaves on the way from the zero-position in an end-position the pre-determined measurement plane, the analysis program is able to output a warning. Thus, it is possible to correct a not correctly executed movement immediately and to ensure a correct measurement result.

### 4. SHORT DESCRIPTION OF THE DRAWINGS

In the following, preferred embodiments of the present invention are described in the context of the figures. In which shows:

FIG. 1 a block-diagram of components of an embodiment of a device for measuring mobility of extremities according to the invention;

FIG. 2 a three-dimensional view of an embodiment of a measuring means according the invention for measuring mobility of extremities;

FIG. 3 a three-dimensional exploded view of the embodiment of a measuring means for measuring mobility of extremities according to the invention according to FIG. 2

FIG. 4 a schematic overview of the device for measuring mobility of extremities according to the invention during a measurement;

FIG. 5 a screenshot of an embodiment of the analysis program according to the invention, while an individual measurement profile is combined from a pool of single measurements;

FIG. 6 a screenshot of an embodiment of the analysis program according to the invention, while the zero-position of an elbow-joint of the proband is defined;

FIG. 7 a screenshot of an embodiment of the analysis program according to the invention, during the execution of a measurement of the mobility of the elbow-joint of the proband;

FIG. 8 a screenshot of an embodiment of the analysis program according to the invention, during the output of the measured results of the measurement of a mobility of a hip in the documentation-form according to the neutral-zero-method; and

FIG. 9 a screenshot of an embodiment of an analysis program according to the invention, during the input of subjective assessments during the measurement.

### 5. DETAILED DESCRIPTION OF PREFERRED EMBODIMENTS

In the following, with reference to the figures, preferred embodiments of the present invention are described in detail. Single features of specific embodiments can be combined with features of other embodiments, when it is reasonable.

The device for measuring as well as the corresponding method is described in the following in a preferred situation, in which a proband is examined by an examiner, wherein the examiner attaches a measuring means at an extremity of the proband and then guides the extremity of the proband in the desired movement plane. But alternatively, it is also possible, that the movements are carried out by the proband alone.

Furthermore, the device and the method are explained at the example of the neutral-zero-method. Another suitable method for the determination of mobility of extremities can be also supported.

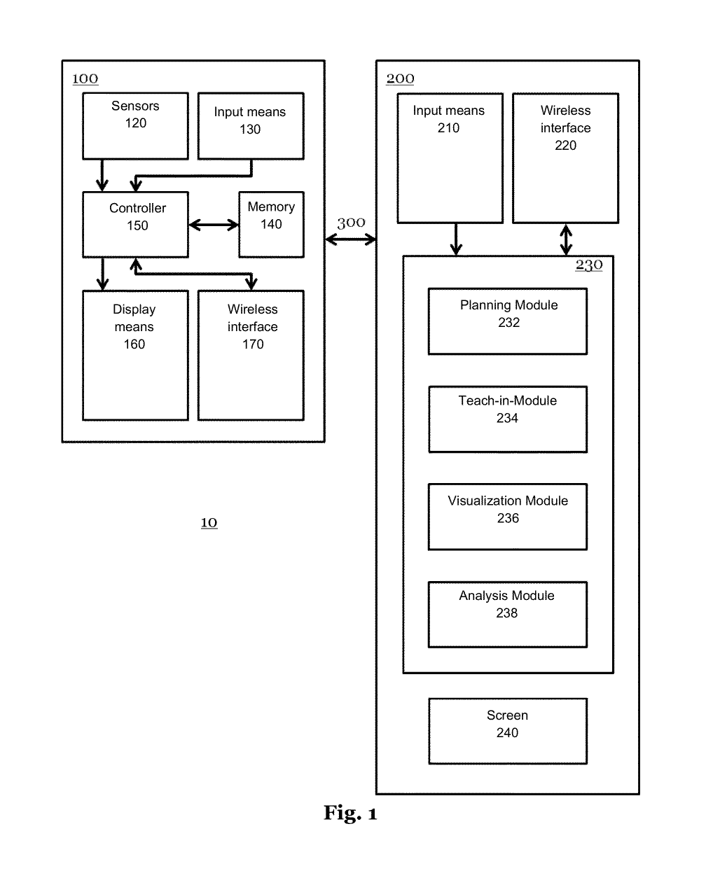

FIG. 1 shows a block-diagram of the components of an embodiment of a device for measuring mobility of extremities 10 according to the invention, which is named in the following also as measuring device 10. The measuring device 10 comprises as main components a wireless measuring means 100, which is attached for the measurement to the extremity, and a data processing device 200 for analysis and documentation. The measuring means 100 and the data processing device 200 are connected to each other in data-matters via a radio link 300. Radio link 300 is preferably bi-directionally designed. That means that data can be transmitted from the measuring means 100 to the data processing device 200 and vice versa. Also a unidirectional radio link 300 is possible, which transmits the measured values from the measuring means 100 to the data processing device 200. As radio standard, for instance Bluetooth, W-LAN or another digital standard can be used.

The sensor data is transmitted wirelessly to the data processing device 200. In the analysis program 230, which is executed there, the analysis of the detected measured values is carried out, wherein relative movements and relative orientations of the extremity to be measured are calculated from the measured values. Thereof, then the mobility of the extremity can be computed for instance by the use of the neutral-zero-method and can be displayed. The analysis can be outputted numerically, graphically or audio-visually via the output means (screen or printer) of the data processing device or via output means of the wireless measuring means.

The measuring means 100 commonly comprises several sensors 120 for the detection of orientation and position. The sensors 120 enclose preferably a three-dimensional magnetic field sensor 122, a three-dimensional gyroscope 124 and a three-dimensional acceleration sensor 126. Other sensors, like for instance gravity field sensors, can be used also. The sensors determine preferably relative orientation- and position-changes, wherein the measurements are determined in parts overdetermined. Herewith, the relative orientation- and position-changes of the measuring means can be computed very precisely.

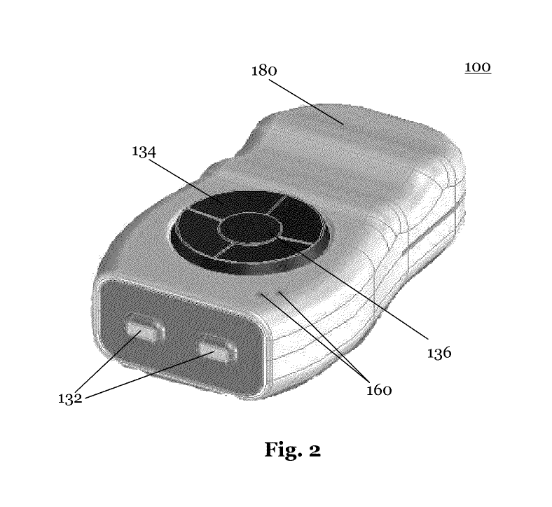

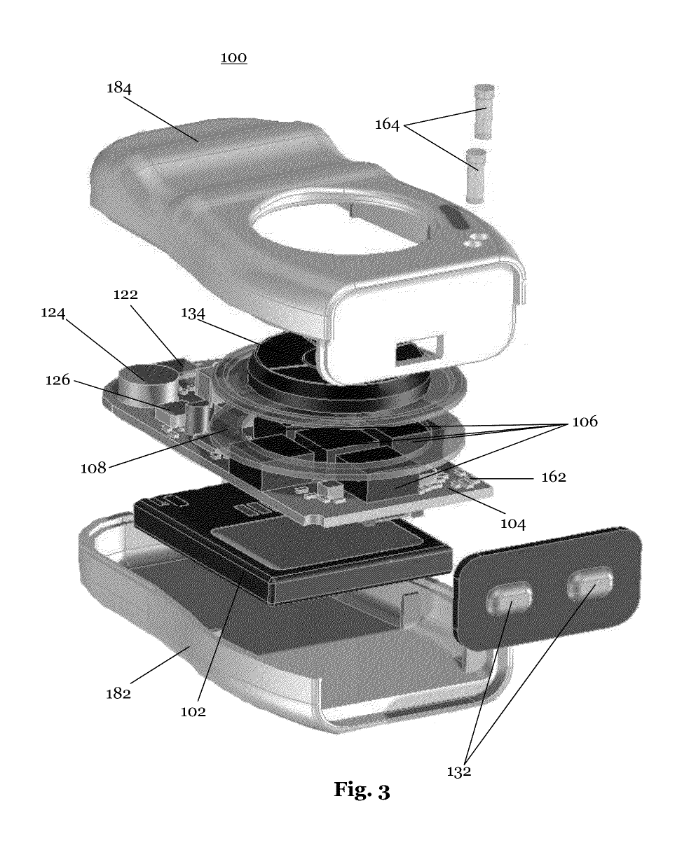

The measuring means 100 furthermore comprises input means 130, by which it is possible to carry out manual inputs concerning the measurements and to control the analysis program 230. The input means 130 can enclose all kinds of buttons and sensors, which are suitable for converting a manual input into an electric signal that can be analyzed by the measuring means 100 itself or by the data processing device 200. As it is shown in FIGS. 2 and 3, the measuring device 100 comprises on the side, which is opposed to the proband a plurality of directional pad pushbuttons 134, which builds together a control cross. Herein, the directional pad pushbuttons 134 represent classically the directions “up”, “down”, “left” and “right”. Other orientations are also possible. Additionally, the directional pad pushbuttons comprise a central pushbutton 136, which can be used for instance for the confirmation of a choice, which is done via the control cross. The pushbutton 136 is also preferably used for the definition of a zero-position and a position of the measurement.

The signals, that are outputted by the sensors 120 and the input means 130 are supplied to a controller 150. Herein, the signals of the sensors 120 can be analog or digital signals. The controller converts the signals when necessary from an analog in a digital signal, stores these signals where appropriate in a memory 140, carries out interim calculations, transmits the measured values by means of a wireless interface 170 via the radio link 300 to the data processing device 200.

The measuring means 100 can also comprise indication means 160 in order to suggest specific operation conditions (for instance empty accumulator 120) or the correct connection via the radio link 300. The indication means may also serve to give an optical feedback of carried out inputs to the user. In addition, the measuring means can acknowledge every input with an acoustic signal. The indication means 160 may comprise luminous diodes 162, LCD-displays or screens of all kinds (not shown).

The measuring means 100 can furthermore comprise fixation means, by which it is fixed at the extremity 22 to be measured of a proband 20. Such fixation means may possibly comprise a hook-and-loop-tape or a belt.

The data processing device 200 encloses preferably a commercial computer or a commercial portable notebook. The data processing device 200 comprises common input means 210, like for instance a keyboard, a computer mouse or a touch-sensitive surface of a screen. Via these input means 210, it is amongst others possible to control the analysis program 230.

The analysis program 230 comprises an analysis module 238, that computes from the measured values of the measuring means 100 position- and orientation-changes of the measuring means 100 and derives thereof a mobility of an extremity. The analysis program receives and computes besides the measured values also the manual inputs of the user via the input means 130, 210.

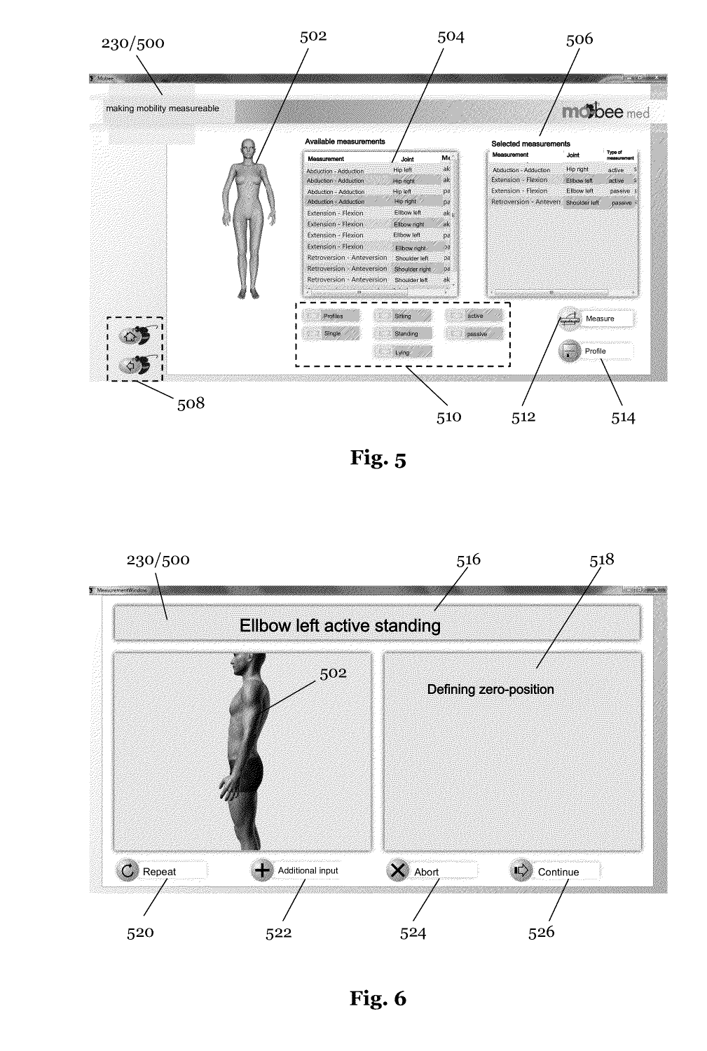

Furthermore, the analysis program 230 comprises a planning module 232 that serves for the preparation of the measurements to be executed. The preparation can consist of a definition of one or more single measurements or of a selection of single measurements out of a pool 504 of single measurements. These can be combined to an individual measurement profile 506 for these specific proband, like it is shown in FIG. 5. These measurement profiles 506 can be also stored and reused later on.

Furthermore, the analysis program 230 comprises a teach-in-module 234 that serves for defining the measurement plane, in which the following the measurement shall take place. Herewith, in succession, the movement sequence can be supervised during the execution of the measurement and possible mistakes can be signalized directly to the examiner.

A visualization module 236 of the analysis program 230 supports the proband 20 and the examiner during the execution of the measurement by graphically screening the movement to be carried out before the measurement. Furthermore, the data, which is transmitted by the measuring means 100 during the measurement, is analyzed by the analysis module 238 in real time and the current position of the extremity is visualized online in the analysis program 230.

An analysis module 238 of the analysis program 230 receives the raw data that is transmitted from the measuring means 100 and computes thereof the current relative position and relative orientation of the measuring means 100. This relative position and relative orientation is used on the one hand by the visualization module 236 in order to display in real time the current position and orientation of the extremity. Furthermore, after the completion of the measurement, the analysis module 238 computes the mobility of the extremities of the proband in form of the desired representative indicators, in particular as number triples 534 according to the above described neutral-zero-method.

A screen 240 of the data processing device 200 serves for the graphical display of the outputs of the analysis program 230.

FIGS. 2 and 3 show a three-dimensional view of an embodiment of a measuring means 100 according to the invention for measuring mobility of extremities, wherein FIG. 2 shows an entire view and FIG. 3 an exploded view.

The measuring means 100 comprises a housing 180, consisting of a lower shell 182 and an upper shell 184. The housing 180 protects the inside arranged components and serves for the simple cleaning and disinfection of the part of the measuring device 100, which gets in contact with the proband.

Through the upper shell 184 of the housing 180, the manual input means 130 go through. The shown input means 130 comprise a front push-button 132, which can be activated by the examiner also then, when the measuring means 100 is attached and fixed by a hand of the examiner at the extremity 22 of the proband 20. Furthermore, the input means comprise a control cross, consisting of directional pad push-buttons 134 and a central push-button 136.

The manual input means 130 serve for the hygienic and comfortable control of the analysis program 230 and of the actual measurement. If hygiene- and comfort-aspects are less relevant, the analysis program 230 can be controlled during the preparation and the analysis of the measurement also via the common input means 210 of a data processing device 200, like for instance via a keyboard, a mouse or a touch screen.

The measuring means 100 in the embodiment, which is shown in the FIGS. 2 and 3 comprises also indication means 160, which consist of luminous diodes 162 on a control board 104 and corresponding optical wave guides 164. These indication means are used as explained above in order to indicate specific conditions of the measuring means or to acknowledge manual inputs optically.

Besides the luminous diodes 162, the control board 104 furthermore comprises a three dimensional magnetic field sensor 122, a three dimensional gyroscope 124 and a three dimensional acceleration sensor 126, which as explained above provide corresponding measured values for the determination of the relative orientation and position. Additionally, on the control board 104 also a gravity field sensor can be arranged.

Furthermore, on the control board a plurality of push-button sensors 106 is arranged, which converts the pressure impulses of the directional pad push-buttons 134 and of the central push-button 136 into electric signals. The control board 104 is the central element of the measuring means 100 and comprises moreover a controller 150, a memory 140 and a wireless interface 170, which is not shown in FIG. 3.

The measuring means 100 is protected via sealings like for instance a sealing ring 108 against the intrusion of liquids or solid dirt particles. Besides the protection of the electronics this serves in particular for the simplified cleaning and disinfection of the measuring means 100 after a direct contact with the proband 20.

Furthermore, in the housing 180 an energy source 102, like for instance an accumulator or a battery is stored, which supplies the measuring means 100 during the operation with electric energy.

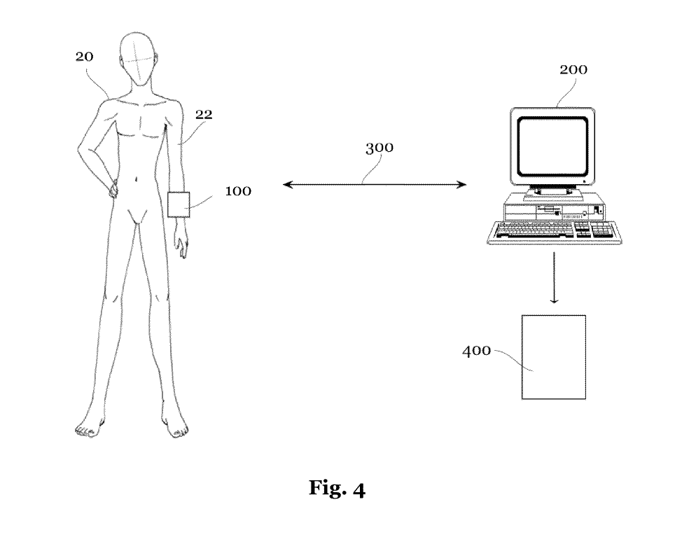

FIG. 4 shows a schematic overview over the cooperation of a proband 20 with an embodiment of the device 10 for measuring mobilities of extremities according to the invention. Herein, the measuring means 100 is fixed to an extremity 22—here at the fore-arm—of the proband 20. The measuring means 100 stays in contact via the radio link 300 with the data processing device 200. In the data processing device 200, the analysis program 230 is executed, which can be operated in particular by the input means 130 of the measuring means 100.

The results of a measurement are preferably outputted as measurement record 400. Its output can be done via a display means 240 of the data processing device 200, here a screen, or as a print via a linked printer (not shown) and can also be digitally stored.

The FIGS. 5-9 show screenshots of a user interface 500 of the analysis program 230 in the different phases “preparation”, “execution” and “analysis” of a measurement of mobilities of extremities. By the use of these figures, in the following the single functions and the use of the analysis program 230 are explained.

As initial situation, a proband 20 shall be examined by an examiner. The analysis program 230 is installed on the data processing device 200 and was started. The user interface 500 of the analysis program 230 is shown on the screen 240 of the data processing device 200. The measuring means 100 is wirelessly connected to the data processing device 200 via the radio link 300.

The examiner controls the analysis program 230 via the input means 130 of the measuring means 100. The analysis program 230 with its user interface 500 is designed in that all inputs, which are necessary for planning, execution and analysis of the measurement can be done easily and fastly via the measuring means 100. Alternatively, the inputs can be also done via the common input means 210 of the data processing device 200.

FIG. 5 shows the analysis program 230 during the preparation of the measurement, in the step of combining an individual measurement profile 506 out of a pool 504 of single measurements. The single measurements are selected via the control cross 134, 136 of the measuring means 100 and are combined to an individual measurement profile 506. In order to facilitate the selection, the single measurements of the pool 504 can be presorted by specific filters 510. Possible categories for such filters 510 are “profile” and/or “single” and respectively “sitting” and/or “standing” and/or “lying” and respectively “active” and/or “passive”.

The user is guided in the analysis program 230 via navigation helps 508. They allow for instance to go back by means of a so called “home-button” to the starting page or to go one step back in the program sequence.

After the finishing of the profile combination, the measurement is started via the field measurement start 512. The profile 506 can be stored before the execution of the measurement also via the profile management 514. It is also possible via the profile management 514, to load an already stored profile 506 in spite of defining a new one. Furthermore it is possible to modify a loaded profile 506 before the measurement is executed, if this is necessary.

FIG. 6 shows a further step of the preparation of a measurement, namely the definition of the zero-position. As already explained above, a measurement according to the neutral-zero-method always starts from a predefined initial position, the so called zero-position. This principal of the predefined initial position is transmitted during the measurement method according to the invention also on the definition of a starting point of a relative coordinate system of the measuring device 10. Since the detected measured values are—as explained above—mostly relative values, the end position and the end orientation of an extremity can be computed via the defined zero-position.

The user interface 500 of the analysis program 230 indicates the current measurement 516 and explains by means of operation instructions 518, which steps have to be done as next. In addition, the analysis program 230 offers the possibility to repeat a measurement via a button 520, to input additional inputs 522, like for instance the pain feeling or the end feeling of the proband, to abort the measurement 524 or to proceed with the next step of the measurement by means of the button 526.

FIG. 7 shows the user interface 500 of the analysis program 230 during the execution of a measurement. A graphical representation 502 of the movement to be carried out facilitates the intuitive understanding of the measurement, which is named in the title 530. Above the window with the representation 502 and the title 530, a navigation bar 528 is arranged, by which the user can switch by means of the arrows left and right between the single measurements of the measurement profile 506. Furthermore, this representation gives a survey over preceding and still coming measurements. Preferably, the graphical representation 502 shows during the measurement in real time the current position and orientation of the extremity 22 to be measured of the proband 20.

In addition, this real time-representation 502 is used in order to detect the movement sequence of the extremity to be measured 22 and possibly to indicate deviations out of the measurement plane visually and/or acoustically.

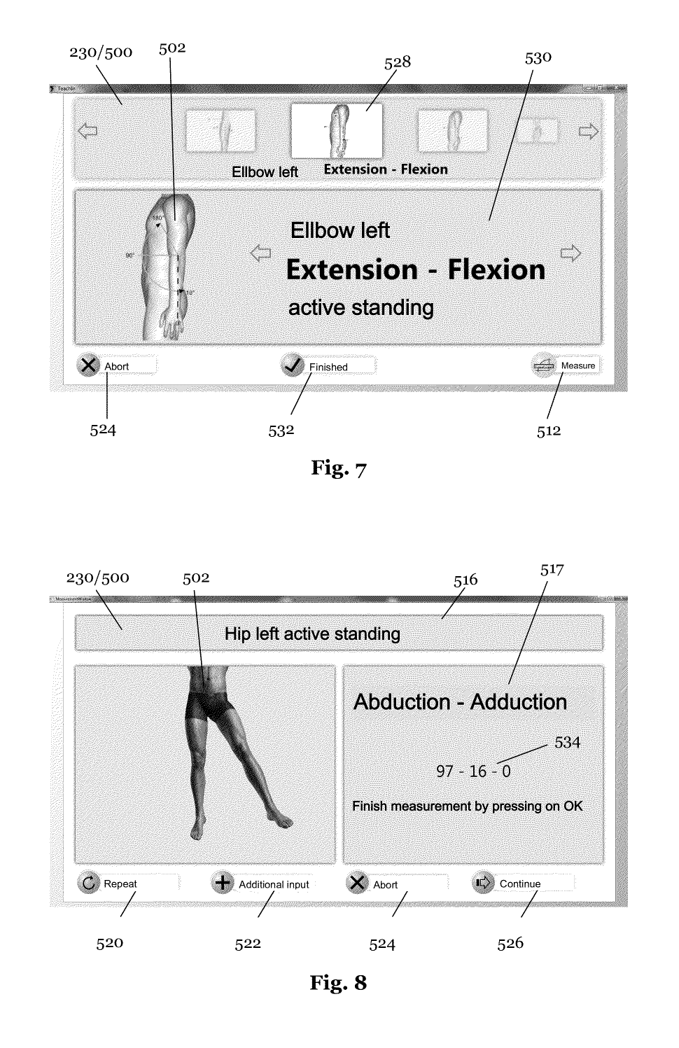

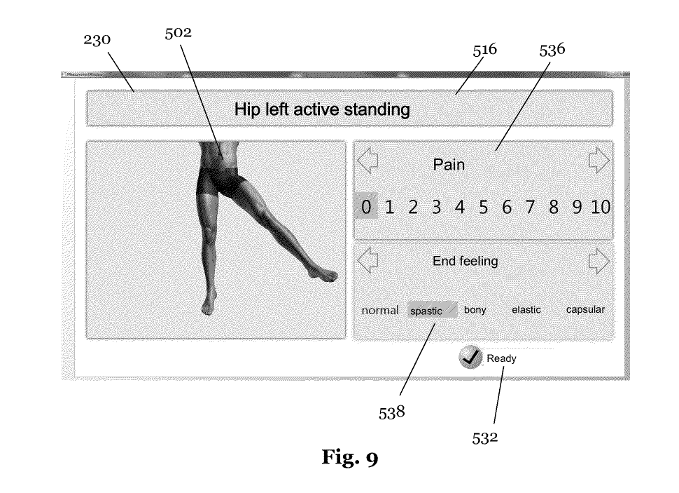

FIG. 8 shows the user interface 500 of the analysis program 230 after the analysis of a measurement. Besides the title 516 and the declaration of the current measurement 517, a graphical representation 502 of the just now executed movement is shown. Preferably, the graphical representation 502 shows the movement, which was detected during the measurement, of the extremity 22 of the proband 20. The actual measurement result is displayed in this preferred embodiment in form of a number-triple 534, according to the neutral-zero-method.

Additionally, the analysis program 230 also offers in the measurement window the possibility to repeat a measurement by means of button 520, to document additional inputs like for instance pain and/or end feeling by means of button 522, to abort the measurement by means of the button 524 or to proceed with the next step by means of button 526.

FIG. 9 shows the user interface 500 of the analysis program 230 during the input of such additional inputs, here “pain” (scale 536) and “end feeling” (scale 538). After the measurement, the user can directly move the selection cursor, i.e. a graphically highlighted field, via the input means 130 of the measuring means 100 leftwards and rightwards and can then select the desired assessment.

After the acknowledgement of the carried out inputs via the finished-button 532, this single measurement is completed and it can be proceeded with the next single measurement of the measurement profile 506. After the last measurement of a measurement profile 506, the analysis program 230 offers several possibilities of data analysis and data storage.

### LIST OF REFERENCE SIGNS

10 device for measuring and assessing mobilities of extremities and of body parts
20 proband
22 extremity
100 measuring means
102 energy source
104 control board
106 push-button sensors
108 sealing rings
110 mounting means
120 sensors
122 three-dimensional magnetic field sensor
124 three-dimensional gyroscope
126 three-dimensional acceleration sensor
130 input means
132 front push-button
134 directional pad push-buttons
136 central push-button
140 memory
150 controller
160 indication means
162 luminous diodes
164 optical waveguide
170 wireless interface
180 housing
182 lower shell
184 upper shell
200 data processing device
210 input means
220 wireless interface
230 analysis program
232 planning module
234 teach-in module
236 visualization module
238 analysis module
240 screen
300 radio link
400 measurement record
500 user interface of the analysis program 230
502 graphical representation
504 list of possible single-measurements/pool
506 measurement profile
508 navigation helps
510 filter
512 measurement start button
514 profile management
516 title of a single-measurement
517 declaration of a single-measurement
518 operation instructions
520 repeated measurement button
522 additional input button
524 measurement abort button
526 measurement continuation button
528 navigation bar
530 single-measurement title
532 finished button
534 measurement result indication
536 subjective assessment pain
538 subjective assessment end-feeling

1. A device for measuring and assessing mobilities of extremities and of body parts of a proband, comprising:
a. a wireless measuring means that is attachable or fixable at an extremity to be measured or at a body part to be measured of the proband, wherein the measuring means comprises at least one sensor for a three-dimensional continuous detection of position changes of the measuring means;
b. a data processing device, that wirelessly receives the measured values of the measuring means;
c. an input means for manual inputs by a user, wherein the input means are part of the wireless measuring means;
d. an analysis program for a planning of a measurement and/or for a visualization of a measurement and/or for an analysis of a measurement; wherein
e. the analysis program runs on the data processing device and the analysis program computes from the measured values of the measuring means position changes of the measuring means;
f. computes from the position changes a mobility of an extremity or of a body part; and
g. processes inputs of the user manually inputted via the input means.

2. A device according to claim 1, wherein the sensor comprises a three-dimensional magnetic field sensor and/or a three-dimensional gyroscope and/or a three-dimensional acceleration sensor in order to detect position- and orientation-changes of the wireless measuring means.

3. A device according to one of the claims 1, wherein the analysis program determines from the position changes all three space coordinates and the attitude of the measuring means.

4. A device according to one of the claims 1, wherein the measured values of the sensors of the position- and orientation-changes are overdetermined.

5. A device according to one of the claims 1, wherein the input means are designed for multi-dimensional inputs, in particular for the navigation in the analysis program or for the input of assessments.

6. A device according to one of the claims 1, wherein the sensor detects the incline of the measuring means and the analysis program computes from the incline manual input values of the user.

7. A device according to one of the claims 1, wherein the sensors do not need any reference points in the measuring field.

8. A device according to one of the claims 1, wherein the manual inputs enclose an identification of a zero-position of the extremity and/or of an end-position of the extremity and/or noting a subjective assessment concerning the proband during the measurement.

9. A device according to one of the claims 1, wherein the analysis program comprises a teach-in-module for the calibration of the measurement plane before executing the real measurement.

10. A device according to one of the claims 1, wherein the analysis program comprises a pool of pre-defined single measurements, which can be combined by the analysis program to a measurement profile.

11. A method for measuring and assessing mobilities of extremities and of body parts of a proband, comprising the following steps:
a. providing a wireless measuring means that is attachable or fixable at an extremity to be measured or at a body part to be measured of the proband, wherein the measuring means comprises sensors for the three dimensional continuous detection of position changes of the measuring means;
b. providing a data processing device, which receives the measured values of the measuring means wirelessly;
c. providing an input means for manual input by a user, wherein the input means is part of the wireless measuring means;
d. attaching or fixing the measuring means at the extremity to be measured or at the body part to be measured of the proband;
e. moving the extremity of the proband;
f. manually inputting a subjective assessment of the proband via the input means; and
g. analyzing the measurement by the analysis program.

12. A method according to claim 11, further comprising a step of choosing the measurement to be executed in an analysis program out of a pool of pre-defined single measurements.

13. A method according to one of the claims 11, wherein the user can navigate in the analysis program by means of the input means of the wireless measuring means or can choose the measurements to be executed.

14. A method according to one of the claims 11, further comprising a teach-in-step, by which the movement plane to be measured is recognized and the analysis program is calibrated.

15. A method according to one of the claims 11, wherein the analysis step comprises a partial step of a continuous computing of the relative-position and -orientation in space out of a plurality of overdetermined measured results of three-dimensional sensors.

16. A method according to one of the claims 11, further comprising a step of numeric and/or graphic real time output of the measuring results during the measurement on a screen of the data processing device.
